(19) United States
(12) Patent Application Publication
(10) Pub. No.: US 2023/0000935 A1
(43) Pub. Date: Jan. 5, 2023

(54) Extract Infused Cannabis Flower, Natural preservation of Cannabis Flower and the method therefor

(71) Applicant: Joseph Anthony Bidwell, Maple Ridge (CA)

(72) Inventor: Joseph Anthony Bidwell, Maple Ridge (CA)

(21) Appl. No.: 17/305,317

(22) Filed: Jul. 3, 2021

(51) Int. Cl. A61K 36/185

(52) U.S. Cl. CPC A61K 36/185; A61K 2236/15; A61K 2236/333; A61K 2236/51

(57) ABSTRACT

Extract Infused Cannabis Flower, The natural preservation of Cannabis flower and the method of infusing and preserving is provided. The method includes saturating cured cannabis flower with alcohol in a container, diluting cannabis extract with alcohol in a separate container, adding the diluted cannabis extract to the saturated cannabis flower, mixing thoroughly, and removing the alcohol by heat evaporation which allows time for the extract to be absorbed completely through the flower.

### CROSS REFERENCE TO RELATED APPLICATIONS

This application is a continuation in part of U.S. Provisional patent Application No. 63/048,061 Filed on 3 Jul. 2020.

### TECHNICAL FIELD

This application relates to cannabis. In particular, it relates to increasing the potency of parts of the cannabis plant, infusing parts of the cannabis plant and preserving parts of the cannabis plant.

### BACKGROUND OF THE INVENTION

The Cannabis industry is growing rapidly around the world. Cannabis flower has a relatively short shelf life and is very inconsistent from flower to flower. In the area of cannabis most enjoy the effects of smoking Cannabis but they also enjoy the taste and smoothness of the inhale. There is a strong need for a well preserved Cannabis Flower product that maintains consistency in THC, Flavour and Smoothness.

### SUMMARY OF THE INVENTION

Cannabis flower can be smoked but there is never a guarantee of freshness or the level of THC. Infusing a cured cannabis flower with an extract from cannabis naturally preserves the Cannabis flower. The Cannabis Flower and Cannabis Extract become one, the extract is absorbed by every part of the flower plant matter including the stem. The extract contains wax's and oil's that have a longer shelf life then Cannabis flower on its own.

The Cannabis extract has a more concentrated level of THC giving the infused Cannabis flower a consistent THC level. The extract has a well defined flavour giving the Infused Cannabis Flower a Consistent Flavour. The Cannabis Extract has a naturally smooth inhale giving the infused Cannabis Flower a consistently smooth inhale.

The finished extract infused Cannabis flower product is similar in texture and colour to cigarette tobacco. The finished extract infused Cannabis flower product is convenient as it is already ground up and ready to be rolled in a paper or placed in a smoking apparatus.

### BRIEF DESCRIPTION OF THE DRAWINGS

The accompanying Drawings are intended to help describe the method of the invention and not to limit the invention to the method shown in the drawings.

FIG. 1: Shows ground cannabis flower saturated with alcohol in a glass container.

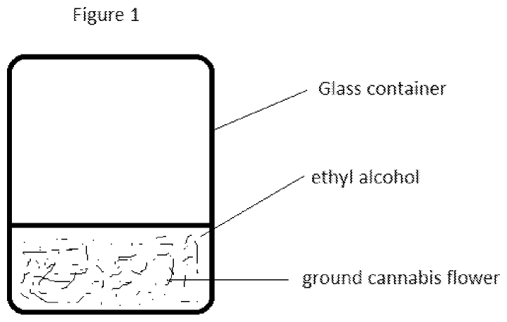

FIG. 2: Shows cannabis extract diluted in alcohol.

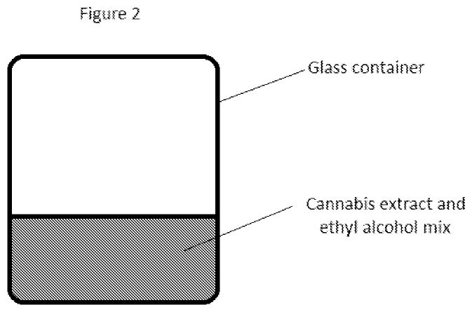

FIG. 3: Shows the cannabis extract and alcohol mix being poured into the container holding the alcohol saturated cannabis flower.

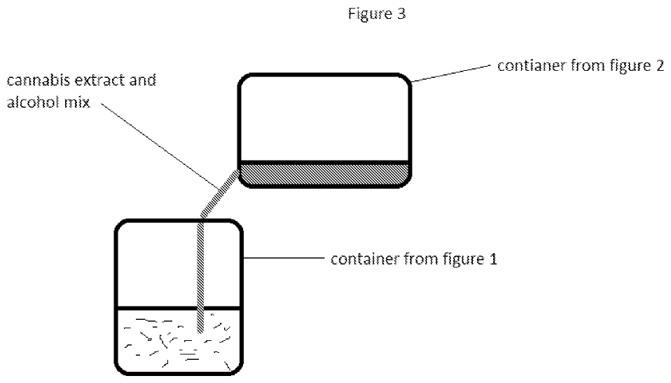

FIG. 4: Shows the cannabis extract, ground cannabis flower and alcohol being stirred until mixed thoroughly.

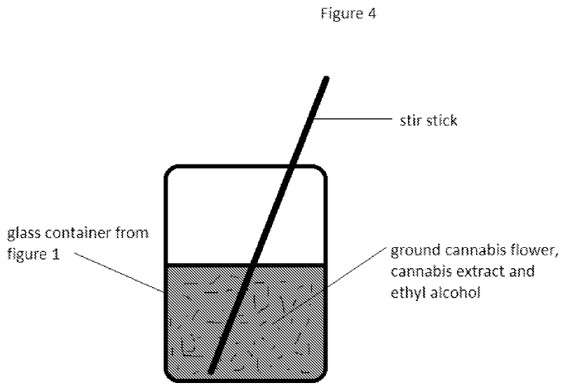

FIG. 5: Shows the container containing the cannabis extract, ground cannabis flower and alcohol on a heat source evaporating the alcohol.

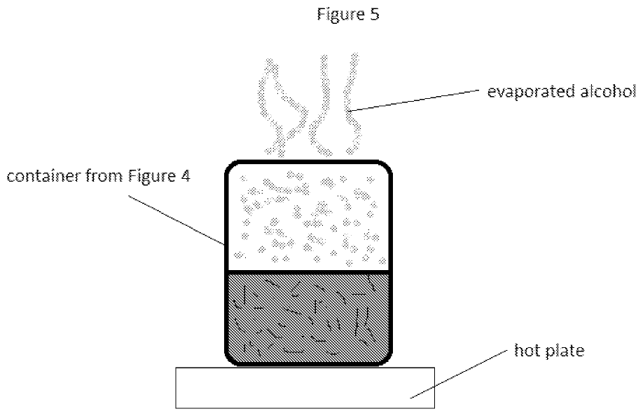

FIG. 6: Shows the container from FIG. 5 pouring out the 99% finished, extract infused cannabis flower product onto a glass tray.

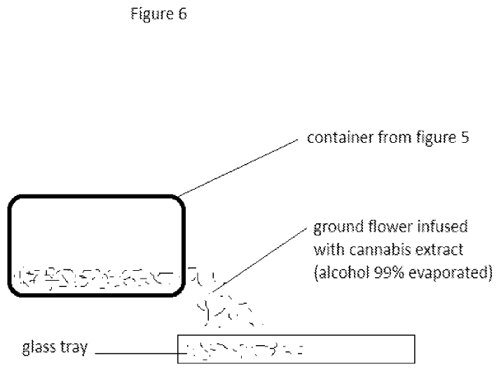

FIG. 7: Shows the glass tray from FIG. 6 being heated in an oven to evaporate the remaining alcohol.

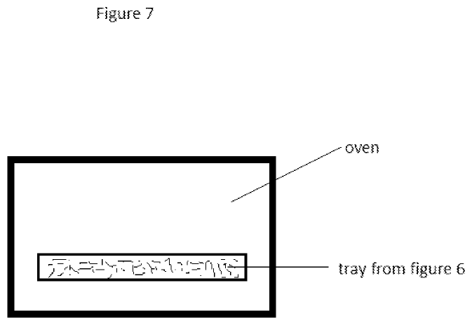

FIG. 8: Shows the glass tray from FIG. 7 holding the finished product, (extract infused and naturally preserved cannabis flower.

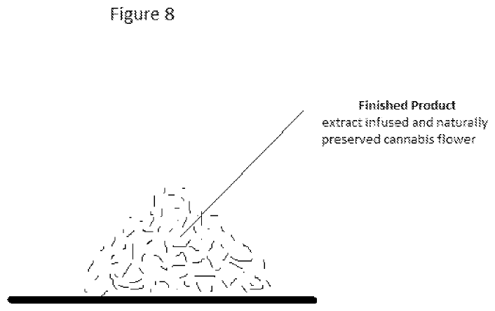

### DETAILED DESCRIPTION OF THE INVENTION

Part A: The Materials Needed are as Follows:

Glass jar with lid (or metal container, plastic container)

Coffee grinder (hand grinder, blender, food processor, scissors, or any other suitable grinding equipment)

Weigh scale

Glass Pyrex dish (or metal dish)

Cannabis flower (or cannabis leaf, or a combination of cannabis leaf and flower)

USP (United States Pharmacopeia) ethyl alcohol (alternatives are 99.9% USP Isopropyl alcohol, USP rubbing alcohol, or ethanol)

Candle warming plate (or hot plate, or any other suitable heat source)

Super critical CO2 extracted cannabis rosin (or CO2 cannabis extract, or THC (Tetrahydrocannabinol), or cannabis distillate)

Part B: Method

Grind up properly cured cannabis flower. The flower cannot be too dry or it will turn to dust. Ideally, it should have the same size and texture and consistency of cigarette tobacco once ground.

Alternately, leave the cannabis flower whole, cut with scissors, grind with a hand grinder or in a blender/food processor. The same can be done with cannabis leaf. It is preferred to dry the flower or leaf to a smokable level, but this is not strictly necessary.

Weigh the desired amount of ground cannabis flower and place into a glass Jar.

Alternately, place in a plastic container or place in a metal container.

Add USP ethyl alcohol to the jar until the ground cannabis flower is completely saturated.

Alternately, add 99.9% USP isopropyl alcohol, USP rubbing alcohol or ethanol.

Put the lid on the jar and shake it vigorously for 10-15 seconds. Alternately, put the lid on the plastic or metal container and shake it vigorously for 10-15 seconds. All shaking steps are optional but preferred.

Shake the jar vigorously for 10-15 seconds twice daily for 3 days. Alternately, shake the plastic or metal container vigorously for 10-15 seconds, twice daily for 3 days.

Weigh out the desired amount of super critical CO2 extracted cannabis rosin. Alternately, one can use other extracts. The formula is 4 parts cannabis flower to 1.65 parts super critical CO2 extracted cannabis rosin. Example: 100 grams of cannabis flower should relate to 41.25 grams of super critical CO2 extracted cannabis rosin.

Alternately, one may use CO2 cannabis extract or THC cannabis distillate or a combination of super critical CO2 extracted cannabis rosin, CO2 cannabis extract and or THC cannabis distillate.

The ratio can be more or less, but may tend to just be gooier, as it stands now with this ratio it comes out ready to roll.

Mix USP ethyl alcohol with super critical CO2 extracted cannabis rosin so the product becomes a pourable liquid, preferably but not necessarily in a 1:1 ratio.

Alternately, mix USP ethyl alcohol with CO2 cannabis extract or THC cannabis distillate or a combination of super critical CO2 extracted cannabis rosin, CO2 cannabis extract and/or THC cannabis distillate so the product becomes a pourable liquid.

Pour the USP ethyl alcohol/super critical CO2 extracted cannabis rosin mix into the jar containing the ground cannabis flower.

Alternately, pour the USP ethyl alcohol/CO2 cannabis extract or THC cannabis distillate, or a combination of super critical CO2 extracted cannabis page 5rosin, CO2 cannabis extract and or THC cannabis distillate mix into the jar or plastic or metal container containing the ground cannabis flower.

Replace the lid and shake the glass jar vigorously for 10-15 seconds then let sit for 24 hours. Alternately, replace the lid and shake the metal or plastic container

vigorously for 10-15 seconds then let sit for 24 hours.

Shake the glass jar vigorously for 10-15 seconds then remove the lid. Alternately,

shake the metal or plastic container vigorously for 10-15 seconds then remove the lid.

Place the glass jar on a candle warming plate to evaporate the USP ethyl alcohol slowly. The temperature should be just warm enough to evaporate the alcohol.

Alternately, place the plastic or metal container over or in a heat source just warm enough to evaporate the USP ethyl alcohol, 99.9% USP Isopropyl alcohol, USP rubbing Alcohol or ethanol.

In other embodiments other methods of evaporation are fine. Note that 24 h steps are helpful but not needed.

Keep the glass jar on the candle warming plate and stir twice daily until approximately 95% of the USP ethyl alcohol has been evaporated and the ground cannabis flower has become oily.

Alternately, keep the metal or plastic container on the heat source and stir twice daily until approximately 95% of the USP ethyl alcohol, 99.9% USP isopropyl alcohol, USP rubbing alcohol or ethanol has been evaporated and the ground cannabis flower has become oily. It is possible to use other evaporative methods such as a water bath, vacuum, etc.

Pour the oily ground cannabis flower into a glass Pyrex dish big enough to spread the ground cannabis flower into an even thin layer. Alternately, pour the oily ground cannabis flower into a metal dish big enough to spread the ground cannabis flower into an even thin layer.

Place the glass Pyrex dish into an oven at low temperature, for example 170 degrees F., until the USP ethyl alcohol has been evaporated. Alternately, place the glass Pyrex dish into an oven at low temperature, for example 170 degrees F. until the 99.9% USP isopropyl alcohol, USP rubbing alcohol or ethanol has been evaporated. In other words, a suitable temperature is near the boiling point of the alcohol being used as solvent.

Remove the Pyrex dish, stir the super critical CO2 extracted rosin infused cannabis flower and allow to cool at room temperature.

Alternately, remove the Pyrex dish, stir the CO2 cannabis extract or THC cannabis distillate or a combination of super critical CO2 extracted cannabis rosin, CO2 cannabis extract and or THC cannabis distillate infused cannabis flower and allow to cool at room temperature.

Place the cooled super critical CO2 extracted rosin infused cannabis flower into a glass jar and shake vigorously for 10-15 seconds, keeping the glass jar sealed allow it to sit for 24 hours.

Alternately, place the cooled CO2 cannabis extract or THC cannabis distillate or a combination of super critical CO2 extracted cannabis rosin, CO2 cannabis extract and or THC cannabis distillate infused cannabis flower into a jar and shake vigorously for 10-15 seconds, keeping the glass jar sealed allow it to sit for 24 hours.

A benefit of shaking at this step is to obtain consistency in the end product, but can omit.

Allow for about 24 hours in the glass jar used to ‘sweat’ the product, which pulls residual moisture out of the product. This is a useful step but optional, as one could extract through other drying or evaporative methods.

The super critical CO2 extracted rosin infused cannabis flower is now ready for packaging.

Alternately, the CO2 cannabis extract or THC cannabis distillate or a combination of super critical CO2 extracted cannabis rosin, CO2 cannabis extract and or THC cannabis distillate is now ready for packaging.

The final product, i.e. the infused/preserved cannabis flower or leaf, may optionally be modified through the addition of flavorants and other additives such as terpenes.

In general, unless otherwise indicated, singular elements may be in the plural and vice versa with no loss of generality.

Throughout the description, specific details have been set forth in order to provide a more thorough understanding of the invention. However, the invention may be practiced without these particulars. In other instances, well known elements have not been shown or described in detail and repetitions of steps and features have been omitted to avoid unnecessarily obscuring the invention. Accordingly, the specification is to be regarded in an illustrative, rather than a restrictive, sense.

Although the present invention has been illustrated principally in relation to cannabis flower, it has application in respect of cannabis leaf.

It will be clear to one having skill in the art that further variations to the specific details disclosed herein can be made, resulting in other embodiments that are within the scope of the invention disclosed. All parameters, dimensions, materials, weights, purities and configurations described herein are examples only and actual values of such depend on the specific embodiment. Accordingly, the scope of the invention is to be construed in accordance with the substance defined by the following claims.

1. A cannabis product, comprised of cannabis extract and cannabis flower or leaf.

2. A cannabis product, comprised of cannabis extract, cannabis flower or leaf and terpenes or flavorants.

3. A method for making cannabis flower or leaf infused with cannabis extract comprised of the steps:
A. saturating ground cannabis flower in alcohol.
B. Diluting Cannabis extract with alcohol.
C. Combining the saturated flower with diluted cannabis extract.
D. Heat evaporating the alcohol leaving an extract infused cannabis flower.

4. A method for making flavoured cannabis flower or leaf infused with cannabis extract comprised of the steps:
A. saturating ground cannabis flower in alcohol.
B. saturating ground cannabis flower in alcohol.
C. Combining the saturated flower with diluted cannabis extract.
D. Adding terpenes or flavorants.
E. Heat evaporating the alcohol leaving Flavoured, extract infused cannabis flower.

5. A method for making naturally preserved cannabis flower or leaf comprised of the steps:
A. saturating ground cannabis flower in alcohol.
B. Diluting Cannabis extract with alcohol.
C. Combining the saturated flower with diluted cannabis extract.
D. Heat evaporating the alcohol leaving a naturally preserved cannabis flower.

6. A method for increasing the potency of cannabis flower or leaf comprised of the steps:
A. Saturating ground cannabis flower in alcohol.
B. Diluting Cannabis extract with alcohol.
C. Combining the saturated flower with diluted cannabis extract.
D. Heat evaporating the alcohol leaving a more potent cannabis flower.
